(19) United States
(12) Patent Application Publication
(10) Pub. No.: US 2022/0071886 A1
(43) Pub. Date: Mar. 10, 2022

(54) SKIN ENGAGING MEMBER FOR RAZOR CARTRIDGE

(71) Applicant: BIC-VIOLEX S.A., ANIXI (GR)

(72) Inventors: Georgios TSIAKATOURAS, ANIXI (GR); Giorgos VLACHOS, ANIXI (GR)

(21) Appl. No.: 17/422,958

(22) PCT Filed: Jan. 14, 2020
(86) PCT No.: PCT/EP2020/050818

§ 371 Date: Jul. 14, 2021

Related U.S. Application Data
(60) Provisional application No. 62/792,590, filed on Jan. 15, 2019.

(51) Int. Cl. A61K 8/81; B26B 21/44; A61Q 9/02

(52) U.S. Cl. CPC A61K 8/8141; A61K 2800/546; A61Q 9/02; B26B 21/443

(57) ABSTRACT

This application relates to a razor cartridge including a housing and a skin engaging member, the skin engaging member including a superabsorbent polymer, wherein the superabsorbent polymer is a swellable carrier being loaded with one or more skin active agents.

### CROSS REFERENCE TO RELATED APPLICATION(S)

This application is a National Stage Application of the International Application No. PCT/EP2020/050818, filed on 14 Jan. 2020, now published as WO/2020/148283, and which claims priority to US provisional application US 62792590 filed on 15 Jan. 2019, entitled “SKIN ENGAGING MEMBER FOR RAZOR CARTRIDGE”, the entire contents of which is incorporated herein by reference.

### FIELD

The present disclosure relates to a razor cartridge including a skin engaging member, wherein the skin engaging member includes a superabsorbent polymer which may encapsulate or may be loaded with skin active ingredient(s) or agent(s).

### BACKGROUND

Shaving aids on razor cartridges that provide lubrication and/or that deliver cosmetic or skin conditioning agents during shaving are known. Such shaving aids typically include a water-insoluble matrix material that provide structural integrity and a water-soluble polymer that provides the lubrication and delivers the incorporated active ingredient during the shave once the water-soluble polymer bearing the active ingredient is dissolved and/or leached out of the water-insoluble matrix upon contact with water.

The addition of various active ingredients or skin care agents into shaving aids has also been attempted. These ingredients include skin active agents such as vitamins, botanical extracts, salts, humectants, fragrances, essential oils, silicon oils, organic oils, waxes, antioxidants, exfoliants, depilatory agents, surfactants, hair and skin conditioning agents, anti-bacterial agents, anti-microbial, anti-irritants, antiseptics, biocides, preservatives, skin cooling and soothing agents, moisturizing and hydrating agents, and skin protectants. The ingredients or agents can be included in the shaving aid to be delivered onto the skin after contact. It has been reported; however, that a substantial amount of the agent or active ingredient may be lost or destroyed during manufacturing.

Indeed, the processing conditions to manufacture shaving aids have the consequence that the contained ingredient(s) or agent(s) hardly survive the temperature and/or high shear forces during such conditions. Some technologies may allow keeping an ingredient active or unaffected but fail to deliver the desired amount of the ingredient or agent during wet shaving. Other encapsulation technologies may be more resilient to processing conditions but may not be safe for use in consumer goods intended to be in contact with skin or are too expensive. For example, the addition of cyclodextrin inclusion complexes and displacing agents has also been attempted.

Thus, there is a need for a razor cartridge including a portion to be contacted with the skin, i.e. a skin engaging member, such as a skin contacting/engaging surface of monolayered or multilayered structure, a shaving aid strip, coating or the like, that is able to encapsulate and retain skin active ingredients or agents during manufacturing and storage without loss or with reduced loss of quantity or alteration of the quality of the ingredients or agents and/or that can also deliver the skin active ingredients during shaving upon contact with skin in a wet environment. In particular, the skin engaging member should be stable at least one week, or specifically at least one month at 25° C. and 60% humidity, specifically at 40° C. and 75% humidity or even specifically for three months at room temperature. For example, stability tests could be performed by verifying the presence of skin active ingredients. Specifically, the skin engaging member according to the disclosure exhibits good lubricating property upon contact with water allowing the razor cartridge to become slippery and to reduce friction during contact with skin.

### DETAILED DESCRIPTION

In an aspect, the disclosure relates to a razor cartridge including a housing and a skin engaging member. The skin engaging member includes a superabsorbent polymer. The superabsorbent polymer includes one or more skin active agents encapsulated/loaded therein, wherein the superabsorbent polymer is a swellable carrier being loaded with one or more skin active agents.

Throughout the present description and claims a “superabsorbent polymer” is defined as a polymer, or mix of polymers, which is adapted to imbibe or absorb and retain fluid, such as water, many times its own weight, for example, at least two times its weight or up to ten times its weight, specifically at least five times its weight or up to ten times its weight. Particularly, superabsorbent polymer may be swellable in water-based systems, i.e. aqueous solutions, or in non-water-based systems, i.e. organic or inorganic liquids, or mixtures thereof. In embodiments, superabsorbent polymer may be a water-swellable and/or water-insoluble, organic or inorganic material capable of absorbing e.g. at least two times its weight in an aqueous solution, particularly containing sodium chloride solution in water.

The superabsorbent polymer may be specifically water-swellable (water-absorbing) but not water-soluble. In particular, the superabsorbent polymer is a hydrophilic polymer. Water-soluble polymers that are common in applications involving lubrication and glideness requirements, cannot be considered as the type of superabsorbent polymers detailed herein. A water-soluble polymer loses its properties and structural state upon contact with water and it does not exist as separate entity from the aqueous solution, since it is completely dissolved.

The superabsorbent polymer may be selected from the group consisting of polyacrylates, such as polyhydroxyethylmethacrylate (PHEMA) or sodium polyacrylate, polyacrylamides, homopolymers or copolymers of N-vinyl lactam, ethylene maleic anhydride copolymers, cross-linked carboxymethylcellulose, polyvinyl alcohol polymers or copolymers, cross-linked polyethylene oxide, and starch grafted copolymers of polyacrylonitrile.

In particular, the polyacrylates may be a homopolymer or a copolymer of (meth)acrylic acid, or its salts, such as alkali metal salts, or its esters, such as polyhydroxyethylmethacrylate (PHEMA). Particularly the salts of homopolymer or a copolymer of (meth)acrylate can be chosen from sodium salts of poly(meth)acrylate, particularly sodium polyacrylate. Particularly the esters of homopolymer or a copolymer of (meth)acrylate can be chosen from esters of poly(meth)acrylate, particularly polyhydroxyethylmethacrylate (PHEMA). According to the present disclosure “(meth)acrylate” means methacrylate or acrylate, and “(meth)acrylic acid” therefore means methacrylic acid or acrylic acid. Therefore “homopolymer or a copolymer of (meth)acrylic acid” is directed to homopolymer or a copolymer of acrylic acid or to homopolymer or a copolymer of methacrylic acid.

In some embodiments, the polyacrylamides may be polyacrylamide polymer or copolymers, such as polyacrylamide or poly(prop-2-enamide).

In particular the superabsorbent polymer is selected from the group consisting of homopolymers or copolymers of polyacrylates, homopolymers or copolymers of polyacrylamides, homopolymers or copolymers of N-vinyl lactam, polyvinyl alcohol polymers or copolymers, more particularly from the group consisting of homopolymers or copolymers of polyacrylates and homopolymers or copolymers of polyacrylamides.

The superabsorbent polymer may also be a crosslinked polymer which is capable of absorbing liquids via swelling, specifically aqueous liquids and acts as hydrogel that is able to retain the liquids in accordance with the general definition of superabsorbent.

This way, specifically the structure of the superabsorbent polymer swells and expands when it comes in contact with water or an aqueous solution.

In another aspect, the disclosure relates to a method for loading active agents in a skin engaging member of a razor cartridge as described herein. To do this the method includes providing a skin engaging member, contacting the superabsorbent polymer with a solution containing one or more skin active agents, loading the superabsorbent polymer with the active agents via swelling, and drying the superabsorbent polymer so as to encapsulate the active agents therein. Since the solution contains one or more skin active agents solubilized or dispersed therein, then the one or more active agents will also be absorbed and incorporated into the structure of the superabsorbent polymer by swelling. When the superabsorbent polymer becomes dry, the one or more skin active agents will thus be encapsulated into the superabsorbent polymer. This way, when the superabsorbent polymer is contacted with an aqueous fluid, such as water, for example, during wet shaving, the active agents will be released. This means that since the skin engaging member of the present razor cartridge includes a superabsorbent polymer loaded with active agents, upon contact with an aqueous fluid such as water, for example during shaving, the superabsorbent polymer swells again, and the one or more skin active agent(s) are released to the skin upon contact of the skin engaging member with the skin.

In addition, the release mechanism of the one or more skin active agents is a gradual release “mechanism” that will be triggered during higher number of shavings, i.e. the skin active agents are progressively released during subsequent shavings. The skin active agents are not released at once so as for the superabsorbent polymer to be depleted from the first use.

The above described encapsulation process, i.e. the initial swelling of the active agents by the superabsorbent polymer further allows the superabsorbent polymer to become slippery due to water further being absorbed when the superabsorbent polymer comes in contact with a surface, e.g. skin during a wet shaving. This slippery property makes the skin engaging member, including the superabsorbent polymer and skin active agents, particularly suitable for shavers since the superabsorbent polymer, being slippery reduces the friction of blade-skin during shaving. The further slipperiness of the superabsorbent polymer allows the skin engaging member to act as a lubricant when the skin engaging member is wetted and comes in contact with the skin. While acting as a lubricant, the superabsorbent polymer simultaneously releases and delivers the one or more skin active agents, without being a water-soluble polymer, i.e. without being dissolved, and consequently it lasts longer.

Throughout the present description and claims the term “skin engaging member” signifies a physical structure which engages skin, e.g. for controlled lubrication and skin management functions during shaving. The skin engaging member may have configurations of any type including, but not limited to, one or more fin or fin-like elements, elongated filaments, protrusions, nubs, or any other texture raised or flat such as logos, designs, or graphics. The skin engaging member may be upstanding or curved, flexible/elastic or rigid/solid depending on its material properties, movably attached or fixedly attached to the razor cartridge, may have planar or non-planar surfaces, may be contiguous, non-contiguous, patterned with any functional or decorative patterns of any shape and form, or any combination thereof.

The skin engaging member is typically made as a part of a razor blade cartridge including at least one razor blade supported in a housing made from a polymer material wherein the polymer material of the housing contributes to at least one skin contacting surface.

The polymer material of the housing may be any suitable polymer as currently used to make razor blade cartridges such as acrylonitrile butadiene styrene (ABS), polystyrene, polyurethanes, Polyethylene Terephthalate (PET) but also may be any vinyl polymers, nylons, carbonate polymers, aliphatic polymers or the like, copolymers and/or mixtures thereof, or any other suitable sustainable or biodegradable materials known for making housing of razor cartridges.

The skin engaging member may be part of the housing or extension of the housing or separate (individual) portion from the housing. Attachment of the skin engaging member to the housing can be permanent and substantially immovable or movable. Alternatively, the attachment can be non-permanent, and the skin engaging member can be replaced for any reason such as customization and/or to extend lifetime of the razor. Attachment of the skin engaging member to the housing can be conducted by different engineering methods such as, for example, snap fit, co-injection, sliding coupling or other similar methods.

The razor cartridge may include a blade unit including one or more blades and the skin engaging member may be placed in proximity to the one or more blade(s). The skin engaging member may typically be located at the trailing portion of the cartridge, i.e. rearwards of the blade edges but it may also be located at the leading portion of the cartridge, i.e. forward of the blade edges or it may be placed at both positions on the cartridge and or laterally.

The skin engaging member may be shaped in a continuous or discontinuous configuration. When the skin engaging member is continuous, the skin engaging member may be applied over the entire surface of the housing of the razor cartridge or the skin engaging member may be applied over only one portion of a surface of the housing. The skin engaging member may be a shaving aid strip or a coating or a skin contacting/engaging surface of monolayered or multilayered structure or combinations thereof.

Particularly, the skin engaging member of the razor cartridge may be provided as a coating or may be in the form of a strip. The skin engaging member may further be in the form of a single strip or a plurality of strips of skin engaging member(s) attached to the surface of the housing cartridge.

The skin engaging member may also be configured on the razor cartridge as a monolayer or multilayer, i.e. a skin contacting/engaging surface of monolayered or multilayered structure.

The skin engaging member may include at least one layer of superabsorbent polymer. In embodiments, the skin engaging member may include two, three or even more layers of the superabsorbent polymer. A multilayer skin engaging member may include layers that have identical or different compositions, i.e. the compositions of each layer may be made of the same or different materials. In some embodiments, the skin engaging member may be shaped in at least two or more layers, wherein each layer may be constructed of different polymers, including the superabsorbent polymer. In other embodiments, the skin engaging member may be shaped in two or more layers, including a top layer made of the superabsorbent polymer loaded with active agents as described herein and one or more layers at the bottom made of any material either with or without superabsorbent properties, such asacrylonitrile butadiene styrene (ABS).

In embodiments, the superabsorbent polymer may be a polymer that may be applied in the form of a coating on top of a polymer forming the housing of the razor cartridge. In these embodiments, the superabsorbent polymer may be considered to constitute a hydrophilic coating on the skin engaging member. In some embodiments, the superabsorbent polymer may be a polymer that constitutes the top layer of a skin engaging member configured as multilayer and arranged in the housing of the cartridge. In these embodiments, the coating may cover all or part of the skin engaging member according to the disclosure.

In embodiments, the skin engaging member may be co-injected along with the housing of the cartridge or the skin engaging member may be applied on any surface of the housing directly. In some embodiments, the skin engaging member may be shaped separately from the housing of the cartridge, in a strip form, and attached to the housing. When the skin engaging member is formed as a separate strip, the skin engaging member may be positioned on one or more areas of the housing, such as in a recess that may be situated in the trailing portion of the cartridge, i.e. rearwards of the blades, or even in a guard bar positioned in the leading portion of the cartridge, i.e. in front of the blades of the cartridge, or even on the cutting portion(s) of one or more blades, or on the top, skin contacting surface of at least one side wall of the housing. In some embodiments, the skin engaging member may be placed on two or more of the aforementioned locations, such as for example the skin engaging member may be placed both behind the blades and in front of the blades. Alternatively, the skin engaging member can be applied on auxiliary components of the razor, such as, for example, skin adaptors extending around the housing of the cartridge.

In embodiments, the skin engaging member may be a shaving aid strip or a coating or a monolayered or multilayered structure that are separate (individual) portions from the housing. In embodiments, the skin engaging member may be a skin contacting/engaging surface of monolayered or multilayered structure that is part of the housing or extension of the housing.

In some embodiments, the polymer making the housing of the razor cartridge is bonded directly with a monomer used for the production of the superabsorbent polymer, so that the superabsorbent polymer is directly coated on the housing of the cartridge, thus forming the skin engaging member.

In some embodiments, the superabsorbent polymer may need to be bonded with another polymer or polymer base, different from the material of the housing. In these cases, the skin engaging member including the superabsorbent polymer and the polymer base, is attached to the housing for example through injection, i.e. the polymer base may be co-injected with the plastic of the housing and the superabsorbent polymer may further be manufactured as described below.

In some embodiments, the skin engaging member may be an extruded strip with a polymer base and the superabsorbent polymer may be provided on top as a coating, wherein the skin engaging member may be attached to the housing via glue, ultrasonic weld or snap-fit.

For the construction of the skin engaging member, that has the superabsorbent polymer as a coating on top, a first polymer as a polymer base may be used, which can be, for example, polymers or copolymers consisting of amide and ether groups, e.g. polyether block amides, such as the commercially available as Vestamid® from Evonik Industries AG, or as PEBAX® thermoplastic elastomers from Arkema Industries.

In embodiments, the superabsorbent polymer may be a polymer resulting from a vinyl monomer with hydrophilic properties, such as acrylic acid, acrylamide, 2-hydroxyethyl methacrylate or vinyl alcohol that is grafted on the surface of the polymer base, which is already activated so as to have functional groups for bonding with the vinyl monomer. The vinyl monomer, such as acrylic acid, acrylamide, 2-hydroxyethyl methacrylate or vinyl alcohol is then polymerized to create a polymer coating, which is hydrophilic. This polymer coating acts as the superabsorbent material, for example, it acts as the superabsorbent polymer that can swell the one or more skin active agents and/or cosmetic ingredients dissolved or dispersed in an aqueous solution substantially as disclosed herein, because the superabsorbent polymer has hydrophilic properties. These steps are disclosed in the European Patent No. EP2576673, incorporated herein by reference. The superabsorbent polymer may be poly(meth)acrylic acid or its salts or its esters, polyacrylamide, a polymer that acts as hydrogel in water, such as Polyhydroxyethylmethacrylate (PHEMA), or poly(vinyl) alcohol.

In the context of the present disclosure, the superabsorbent polymer of the skin engaging member tends to swell specifically when contacted with water. Due to these swelling properties, the one or more skin active agents can be incorporated into the superabsorbent polymer. Swelling makes the superabsorbent polymer structure to be more receptive for the penetration of the one or more skin active agents that may be diluted, solubilized, suspended or dispersed into an aqueous solution used to facilitate the swelling.

In some embodiments, the one or more skin active agents can be water-soluble since during the loading of the skin engaging member water is used as solvent. The principle is the active agent be solubilized or dispersed in the water, which is used as solvent or dispersing mean. When the one or more skin active agents are water-soluble, the one or more skin active agents can be solubilized in a water-based medium. The water-based medium serves as a vehicle for the soaking and swelling of the superabsorbent polymer of the skin engaging member. As such, the superabsorbent polymer swells and absorbs water with the one or more skin active agents contained therein.

Consequently, the one or more skin active agents are transferred into the swelled superabsorbent polymer and encapsulated in the structure of the polymer. Once the water of the superabsorbent polymer is dried away, the one or more skin active agents remains encapsulated within the superabsorbent polymer.

In some embodiments, the skin active agents may be not water-soluble. In these cases, it may be necessary to disperse, suspend, or emulsify the one or more skin active agents in the water-based medium. Therefore, the one or more skin active agents may be contained or encapsulated in any vehicle such as micro and/or nanoparticles, micelles, liposomes.

In some embodiments, the non-water-soluble skin active agents may be emulsified, i.e. dispersed as a fine dispersion of minute droplets of one liquid in water-based solution in which it is not soluble or miscible. The emulsion may be micro or nanoemulsion that is stabilized due to the aid of surfactants or the like. In more embodiments, the non-water-soluble skin active agent may be encapsulated within a water-soluble material acting as a shell. This water-soluble material may have specific solubility properties or may be liposomes or niosomes, or any other convenient encapsulation system.

When the one or more skin active agents are not water-soluble but are also not hydrophobic, the one or more skin actives agent may be dispersed as fine particles suspended in the water-based solution that is used for swelling the superabsorbent polymer.

In these embodiments, the viscosity of the water-based solution may be adjusted in order to maintain the particles in suspension and to prevent sedimentation.

The dispersion of the one or more skin active agents may be in the form of micro-micelles, stabilized using surfactants, by encapsulating the active agent, or by any method known to the person skilled in the art to make the one or more skin active agents capable of being water dispersible.

The one or more skin active agent(s) may also be selected from the group consisting of vitamins, botanical extracts, salts, humectants, fragrances, antioxidants, exfoliants, depilatory agents, surfactants, hair and skin conditioning agents, beard softeners, anti-bacterial agents, anti-microbial, anti-irritants, antiseptics, biocides, preservatives, skin cooling and soothing agents, moisturizing and hydrating agents, skin protectants, colorants, film formers, astringents, medicinal agents, soap base, emollients, processing thickening agents from the list of silica, fume silica, TiO2 particles, and combinations thereof.

In particular, the one or more active agent(s) may be selected from the group consisting of botanical/plant oils, essential oils, botanical/plant extracts, vitamins, chamomile and its constituents, botanical butters, polyols, hyaluronic acid, panthenol, allantoin, caffeine, dipotassium glycyrrhizinate (DPG), menthol and menthol derivatives such as menthyl lactate, monomenthyl succinate, and combinations thereof.

Specifically, the one or more active agent(s) selected from the botanical/plant oils may be calendula oil, argan oil, olive oil, almond oil, jojoba oil, and combinations thereof. The one or more active agent(s) selected from the essential oils may be eucalyptus oil, tea tree oil, and combinations thereof. The one or more active agent(s) selected from the botanical/plant extracts may be cucumber extract, aloe vera extract, jojoba extract, plant milks such as coconut milk, and combinations thereof. The one or more active agent(s) selected from the vitamins may be vitamin E. The one or more active agent(s) selected from the chamomile and its constituents may be chamomile extract, alpha-bisabolol, and combinations thereof. The one or more active agent(s) selected from the botanical butters may be Shea butter, cocoa butter, and combinations thereof. The one or more active agent(s) selected from the polyols may be glycerin.

The list of active agents is not limited to the aforementioned, but the active agents may be any compound that can be loaded onto the superabsorbent polymer according to the loading mechanism described above.

The quantity of the one or more skin active agents within the superabsorbent polymer may vary, depending on several parameters, such as the concentration of the one or more skin active agents in the aqueous solution where the agents are dissolved or dispersed (c_{F}), the individual concentration of each one of two or more skin active agents in case of a mixture/formulation of two or more skin active agents being dissolved or dispersed in the aqueous solution (cᵢ), the loading capacity of the superabsorbent polymer to absorb one skin active agent or the mixture/formulation of two or more skin active agents (L), and the absorption ability of one skin active agent within the superabsorbent polymer (aᵢ).

The loading capacity of the superabsorbent polymer reflects the weight of water that can be absorbed from the superabsorbent polymer, e.g. the absorbed water can be at least 2 times the weight of the superabsorbent polymer. For example, the loading capacity can be between 2-10, specifically between 5-10.

The measurement of the quantity of the one or more skin active agents within the superabsorbent polymer may further be correlated with the weight of the superabsorbent polymer per se. For example, the weight of a superabsorbent polymer may be between 5-250 mg, more specifically 10-70 mg, e.g. 30 mg, in order to be loaded sufficiently with skin active agents.

The calculation of the quantity of a skin active agent loaded in the superabsorbent polymer takes into account the aforementioned parameters and follows the equation:

W(AAᵢ)=L×W×c_{F}×cᵢ×αᵢi=1 . . . N (Eq. 1)

### Where:

W(AAᵢ): Weight (in mg) of one skin active agent AAᵢ (i=1−N) loaded in the superabsorbent polymer.

L: Loading capacity of the superabsorbent polymer.

W: Weight (in mg) of the superabsorbent polymer before the loading of one or more skin active ingredients.

c_{F}: Concentration, in % w/w, of the one or more skin active agents in the aqueous solution where the agents are dissolved or dispersed. In embodiments, this is the concentration in % w/w of the mixture/formulation of two or more skin active agents dissolved and/or dispersed in aqueous solution.

cᵢ: individual concentration, in % w/w, of each one skin active agent in the aqueous solution, in case of a mixture/formulation of skin active agents.

aᵢ: absorption ability of the skin active agent, in %, within the superabsorbent polymer. This absorption ability represents, in %, the quantity of active agent that the polymer can absorb from the aqueous solution. This parameter may vary from 100%, which means that almost the totality of the active agent contained in the aqueous solution has been absorbed by the polymer, to 0.0001%, which means that only a very few part of the active agent has migrated within and has been absorbed by the polymer.

This absorption ability can be measured/evaluated by (chromatographic) methods known to the skilled person depending on the nature of the active ingredient and/or of the polymer. Such methods can be monographs, guidelines, databases and information about analytical methods from the World Health Organization, scientific journals, literature, pharmacopoeias, producers and suppliers and the European Medicines Agency. The analytical extraction of such chemicals prior to analysis, can be performed with liquid extraction using organic solvents as described by the relevant method like ethanol, or even binary systems like hexane-water or other well-established extraction methods like solid phase extraction and others.

N: amount of skin active agents. N can be any integer number, for example 1,2,3,4,5...

Consequently, the total weight of two or more skin active agents that are loaded as mixture/formulation in the superabsorbent polymer may be calculated following the equation:

$$W ( A A ) = {\sum }_{i = 1}^{i = N} W ( A {A}_{i} ) , i = 1 … N \qquad ( Eq . 2 )$$

For example, the quantity of each one skin active agent (W_{AAi}) measured individually may have high variations depending on the properties of the superabsorbent polymer and/or the properties of the aqueous solution and of the skin active agents per se.

The quantity/weight of one or more skin active agents (W(AAᵢ))can be calculated from the aforementioned calculations, considering that the parameters of Eq. 1 can be L equal to any value between 2-10, W equal to any value between 5-250 mg, c_{F} equal to any value between 0.0001-80%, cᵢ equal to any value between 0.0001-100%, aᵢ equal to any value between 0.0001-100%.

For example, the quantity of the one or more skin active agents may be from about 0.00000000000000001 mg to about 2 g (2000 mg) within the superabsorbent polymer. In some embodiments, the quantity of the one or more skin active agents is from about 0.001 mg to about 1.5 g (1500 mg) within the superabsorbent polymer.

Example 1: Loading of 1 skin active agent (AA) (cᵢ=100%)

One skin active agent (cᵢ=100%), e.g. chamomile is added in powder form in a solution of deionized water in concentration c_{F}=30% w/w. In the meantime, the skin engaging member including the superabsorbent polymer is dipped for two minutes in another solution of deionized water for swelling to be activated. The superabsorbent polymer is polyacrylamide polymer. Subsequently, the skin engaging member with the superabsorbent polymer is immersed in 200 ml of the aqueous solution containing the skin active agent, e.g. chamomile, in concentration c_{F}=30% w/w. The skin engaging member remains in the aqueous solution containing the skin active agent at room temperature for 24 h. Afterwards, the skin engaging member is collected from the solution and it undergoes drying for 2 h. The absorption ability of the skin active agent in the superabsorbent polymer is estimated to be a₁=5%. The loading capacity of the superabsorbent polymer is estimated to be L=6 and the weight of the superabsorbent polymer (before loading) is 35 mg. Thus, the weight of the skin active agent within the superabsorbent polymer can be calculated using Eq. 1 and considering c_{F}=30% w/w, c₁=100%:

W(AA₁)=6×35×0.3×1×0.05=3.15 mg

### Example 2

According to Example 1, further experiments are conducted in order for other skin active agents to be examined. In a second example, skin active agent such as caffeine is loaded in a superabsorbent polymer, such as polyacrylamide, following the process described in Example 1.

### Example 3

In a third example, skin active agent such as aloe vera is loaded in a superabsorbent polymer, such as polyacrylamide, following the process described in Example 1.

### Example 4

In a fourth example, skin active agent such as dipotassium glycyrrhizinate (DPG) is loaded in a superabsorbent polymer, such as polyacrylamide, following the process described in Example 1.

That way, skin engaging members including superabsorbent polymer that retain skin active agents such as chamomile, caffeine, aloe vera and dipotassium glycyrrhizinate are prepared. The skin engaging members are subsequently incorporated in razor cartridges.

Evaluation of the skin engaging members

The protocol followed in Example 1 for the construction of a skin engaging member retaining chamomile was successfully reproduced in the embodiments for the construction of skin engaging members retaining caffeine, aloe vera, dipotassium glycyrrhizinate (DPG). Upon analysis of the skin engaging members, it is verified that the skin active agents are not destroyed or degraded. As a result, it has been observed upon use with water that the skin engaging members are slippery, have good lubricating properties and provide concurrently skin benefits dependent on the properties of the retained skin active agents. These evaluations have been conducted by expert panels.

Additionally, loading of the one or more active agents by swelling involves a cold process which means that the one or more skin active agents are not destroyed or affected by the applied encapsulation process, i.e. the swelling of the active agents by superabsorbent polymer. This provides for higher concentrations of the one or more skin active agents being loaded into the superabsorbent polymer as high temperature no longer damages active agents.

As explained herein, once the skin engaging member is loaded with the one or more skin active ingredients, the skin engaging member is dried in order that the water is removed and the one or more skin active agents remain “encapsulated” within the superabsorbent polymer and are ready to be released when the superabsorbent polymer of the one or more skin active agent is wetted again, for example, upon contact with the skin during wet shaving.

The loading and release of the one or more skin active agents are simply conducted through contact with water and directly from the superabsorbent polymer per se, without any specific structural configuration of the skin engaging member.

As explained herein, the loading for the skin active agents is based on swelling of the superabsorbent polymer and impregnation of the skin active agents therein. Particularly, the loading is based on the swelling of the superabsorbent material having water-absorbing properties that allows the water with the one or more skin active agents dissolved or dispersed to penetrate therein.

As further explained herein, once the razor cartridge is contacted with an aqueous environment, for example, during wet shaving, the skin engaging member swells again and releases the one or more skin active agents upon contact with the skin. The one or more skin active agents are already encapsulated (by swelling) within the superabsorbent polymer after drying of the superabsorbent polymer. The release of the one or more skin active agents is activated during use of the razor cartridge, e.g. during wet shaving. The presence of the water along with the abrasion induced in each shaving stroke through contact with the skin contribute to the release of the one or more skin active agents.

In embodiments, the method of loading active agents in a skin engaging member may further include at least one of the steps of extrusion, injection-molding, laminating or compression-molding to form the skin engaging member.

In embodiments, the skin engaging member may be manufactured first and then attached onto the razor cartridge.

In order to load the superabsorbent polymer with active ingredients the superabsorbent polymer may be immersed into an aqueous solution that triggers the swelling action.

In embodiments, the skin engaging member may be brought into contact with a solution or a dispersion containing the desired skin active agent(s) or combinations thereof. The concentration of the skin active agent(s) in the aqueous solution can vary from 0.0001% up to 80% by weight of the aqueous solution. The skin engaging member may be immersed into the solution or the dispersion having the superabsorbent polymer already in a swelled condition or the superabsorbent polymer may swell while being immersed in the solution. The structure of the polymer loads the one or more skin active agents inside the superabsorbent polymer until it reaches an equilibrium and the active agents are considered as encapsulated within the superabsorbent polymer.

Absorption of the active agents into the superabsorbent polymer may take place between 15° C. and 50° C., more specifically at room temperatures, for a period of time between 10 minutes to 24 hours. The loading of the one or more skin active agents is quantified and reflected by the measurement of the quantity of the one or more skin active agents in the superabsorbent polymer. Thus, as described herein the loading may vary depending on the parameters that affect the calculation of the quantity of the one or more skin active agents as explained herein. The weight of the skin active agent in the superabsorbent polymer can vary from about 0.00000000000000001 mg to about 2 g (2000 mg) within the superabsorbent polymer.

Thus, the disclosure also relates to a method for loading skin active agents in a skin engaging member of a razor cartridge including a housing and the skin engaging member including a superabsorbent polymer wherein the superabsorbent polymer is a swellable carrier and is loaded with the one or more skin active agents. The method includes the steps of:

providing a skin engaging member

depositing a superabsorbent polymer on all or part of the skin engaging member,

contacting the superabsorbent polymer with an aqueous solution containing one or more skin active agents,

loading the superabsorbent polymer with the one or more skin active agents via swelling, and

drying the superabsorbent polymer so as to encapsulate the one or more skin active agents therein.

As mentioned above, the one or more skin active agents may be soluble in the water-based solution or it may be dispersed or emulsified in the water-based solution.

In embodiments, the step of contacting the superabsorbent polymer with a solution may be achieved by spraying, dipping, soaking or brushing and/or any other suitable methods.

In embodiments, the step of contacting the superabsorbent polymer with a solution may involve a duration of between 10 minutes and 24 hours.

In embodiments, the step of contacting the superabsorbent polymer with a solution may be conducted between 15° C. and 50° C., for example at room temperature.

In embodiments, the step of drying is achieved under conditions leading to a water concentration of between 0.01-4% by weight of the superabsorbent polymer. Particularly, the step of drying is considered complete, when the water concentration in the superabsorbent polymer is measured around 1% by weight of the superabsorbent polymer.

The razor cartridge according to the disclosure is of the type that is used with a hair removal device such as razor tool or depilatory tool. The razor cartridge may be removably connected to the hair removal device or integrally connected to the hair removal device.

Thus, another object of the disclosure is a hair removal device, wherein the hair removal device is of the wet shave type. The hair removal device includes a razor cartridge including a housing and a skin engaging member including a superabsorbent polymer, wherein the superabsorbent polymer is a swellable carrier being loaded with one or more skin active agents, and the cartridge further including a blade unit, wherein the skin engaging member is adjacent to the blade unit.

As is evident from the text presented above, as well as the embodiments below, a variety of embodiments are contemplated. Embodiments encompassed by the disclosure are as follows:

1. A razor cartridge including a housing and a skin engaging member, the skin engaging member including a superabsorbent polymer, wherein the superabsorbent polymer is a swellable carrier being loaded with one or more skin active agents.

2. The razor cartridge of embodiment 1, wherein the superabsorbent polymer is water-swellable and/or is water-insoluble.

3. The razor cartridge of any of embodiments 1-2, wherein the superabsorbent polymer is selected from the group consisting of homopolymers or copolymers of polyacrylates, homopolymers or copolymers of polyacrylamides, homopolymers or copolymers of N-vinyl lactam, ethylene maleic anhydride copolymers, cross-linked carboxymethylcellulose, polyvinyl alcohol polymers or copolymers, cross-linked polyethylene oxide, and starch grafted copolymers of polyacrylonitrile.

4. The razor cartridge of any of embodiments 1-3, wherein the superabsorbent polymer is a polyacrylamide, such as polyacrylamide or poly(prop-2-enamide).

5. The razor cartridge of any of embodiments 1-4, wherein the one or more skin active agents being selected from the group consisting of vitamins, botanical extracts, salts, cooling agents, humectants, fragrances, antioxidants, exfoliants, depilatory agents, surfactants, hair and skin conditioning agents, beard softeners, anti-bacterial agents, anti-microbial, anti-irritants, antiseptics, biocides, preservatives, skin cooling and soothing agents, moisturizing and hydrating agents, skin protectants, colorants, film formers, astringents, medicinal agents, soap base, and emollients.

6. The razor cartridge of any of embodiments 1-4, wherein the one or more skin active agents being selected from the group consisting of chamomile, aloe vera, vitamin E, Shea butter, cocoa butter, cucumber, jojoba, calendula, argan oil, olive oil, eucalyptus, tea tree oil, coconut milk, glycerin, almond oil, hyaluronic acid, panthenol, allantoin, caffeine, dipotassium glycyrrhizinate (DPG), menthol and menthol derivatives such as menthyl lactate, monomenthyl succinate, and combinations thereof.

7. The razor cartridge of any of embodiments 1-6, wherein the weight of the one or more skin active agents is calculated by using the equation W(AAᵢ)=L×W×c_{F}×cᵢ×aᵢ and defining the parameters L equal to any value between 2-10, W equal to any value between 10-100 mg, c_{F} equal to any value between 0.0001-80%, cᵢ equal to any value between 0.0001-100%, aᵢ equal to any value between 0.0001-100%.

8. The razor cartridge of any of embodiments 1-6, wherein the weight of the one or more skin active agents in the superabsorbent polymer is from about 0.00000000000000001 mg to about 2 g (2000 mg) within the superabsorbent polymer.

9. The razor cartridge of any of embodiments 1-8, wherein the skin engaging member is configured as monolayer or multilayer.

10. The razor cartridge of any of embodiments 1-8, wherein the skin engaging member is shaped in a continuous configuration or in a discontinuous configuration.

11. The razor cartridge of any of embodiments 1-8, wherein the skin engaging member is shaped in a continuous configuration and is applied over one portion of the surface of the housing.

12. The razor cartridge of any of embodiments 1-8, wherein the skin engaging member is provided as a single strip or a plurality of strips attached to the surface of the housing of the cartridge.

13. A method for loading active agents in a skin engaging member of a razor cartridge of any of embodiments 1-12, the method including the steps of:

providing a skin engaging member,

depositing a superabsorbent polymer on all or part of the skin engaging member

contacting the superabsorbent polymer with a solution containing one or more skin active agents

loading the superabsorbent polymer with the one or more skin active agents via swelling, and

drying the superabsorbent polymer so as to encapsulate the one or more skin active agents therein.

14. The method of embodiment 13, wherein the step of contacting the superabsorbent polymer with a solution is achieved by spraying, dipping, soaking, or brushing.

15. The method of any of embodiments 13, 14, wherein the step of contacting the superabsorbent polymer with a solution involves a duration of between 10 minutes and 24 hours.

16. The method of any of embodiments 13-15, wherein the step of contacting the superabsorbent polymer with a solution is conducted between 15° C. and 50° C.

17. A hair removal device including the razor cartridge of any of embodiments 1-12, the razor cartridge including a blade unit, wherein the skin engaging member is adjacent to the blade unit.

1. A razor cartridge comprising: a housing and a skin engaging member, the skin engaging member including a superabsorbent polymer, wherein the superabsorbent polymer is a swellable carrier being loaded with one or more skin active agents.

2. The razor cartridge according to claim 1, wherein the superabsorbent polymer is water-swellable and/or is water-insoluble.

3. The razor cartridge according to claim 1, wherein the superabsorbent polymer is selected from the group consisting of homopolymers or copolymers of polyacrylates, homopolymers or copolymers of polyacrylamides, homopolymers or copolymers of N-vinyl lactam, ethylene maleic anhydride copolymers, cross-linked carboxymethylcellulose, polyvinyl alcohol polymers or copolymers, cross-linked polyethylene oxide, and starch grafted copolymers of polyacrylonitrile.

4. The razor cartridge according to claim 1, wherein the superabsorbent polymer is a polyacrylamide.

5. The razor cartridge according to claim 1, wherein the one or more skin active agents being selected from the group consisting of vitamins, botanical extracts, salts, cooling agents, humectants, fragrances, antioxidants, exfoliants, depilatory agents, surfactants, hair and skin conditioning agents, beard softeners, anti-bacterial agents, anti-microbial, anti-irritants, antiseptics, biocides, preservatives, skin cooling and soothing agents, moisturizing and hydrating agents, skin protectants, colorants, film formers, astringents, medicinal agents, soap base, and emollients.

6. The razor cartridge according to claim 1, wherein the one or more skin active agents being selected from the group consisting of chamomile, aloe vera, vitamin E, shea butter, cocoa butter, cucumber, jojoba, calendula, argan oil, olive oil, eucalyptus, tea tree oil, coconut milk, glycerin, almond oil, hyaluronic acid, panthenol, allantoin, caffeine, dipotassium glycyrrhizinate (DPG), menthol and menthol derivatives such as menthyl lactate, monomenthyl succinate, and combinations thereof.

7. The razor cartridge according to claim 1, wherein the weight of the one or more skin active agents is calculated by using the equation W(AAᵢ)=L×W×c_{F}×cᵢ×aᵢ and defining the parameters L equal to any value between 2-10, W equal to any value between 5-250 mg, c_{F} equal to any value between 0.0001-80%, cᵢ equal to any value between 0.0001-100%, aᵢ equal to any value between 0.0001-100%.

8. The razor cartridge according to claim 1, wherein the weight of the one or more skin active agents in the superabsorbent polymer is from about 0.00000000000000001 mg to about 2 g (2000 mg) within the superabsorbent polymer.

9. The razor cartridge according to claim 1, wherein the skin engaging member is configured as monolayer or multilayer.

10. The razor cartridge according to claim 1, wherein the skin engaging member is shaped in a continuous configuration or in a discontinuous configuration.

11. The razor cartridge according to claim 1, wherein the skin engaging member is shaped in a continuous configuration and is applied over one portion of the surface of the housing.

12. The razor cartridge according to claim 1, wherein the skin engaging member is provided as a single strip or a plurality of strips attached to the surface of the housing of the cartridge.

13. A method for loading active agents in a skin engaging member of a razor cartridge according to claim 1, the method comprising the steps of:
providing a skin engaging member,
depositing a superabsorbent polymer on all or part of the skin engaging member
contacting the superabsorbent polymer with a solution containing one or more skin active agents
loading the superabsorbent polymer with the one or more skin active agents via swelling, and
drying the superabsorbent polymer so as to encapsulate the one or more skin active agents therein.

14. The method according to claim 16, wherein the step of contacting the superabsorbent polymer with a solution is achieved by spraying, dipping, soaking, or brushing.

15. The method according to claim 16, wherein the step of contacting the superabsorbent polymer with a solution involves a duration of between 10 minutes and 24 hours.

16. The method according to claim 16, wherein the step of contacting the superabsorbent polymer with a solution is conducted between 15° C. and 50° C.

17. A hair removal device including the razor cartridge according to claim 1, the razor cartridge including a blade unit, wherein the skin engaging member is adjacent to the blade unit.

18. The razor cartridge according to claim 1, wherein the superabsorbent polymer is selected from the group consisting of homopolymers or copolymers of polyacrylates, homopolymers or copolymers of polyacrylamides, homopolymers or copolymers of N-vinyl lactam, polyvinyl alcohol polymers or copolymers.

19. The razor cartridge according to claim 1, wherein the superabsorbent polymer is selected from the group consisting of sodium polacrylate, polyacrylamide or poly(prop-2-enamide).

20. The razor cartridge according to claim 1, wherein the one or more skin active agents being selected from the group consisting of menthyl lactate, monomenthyl succinate, and combinations thereof.
